(19) United States
(12) Patent Application Publication
(10) Pub. No.: US 2005/0153926 A1
(43) Pub. Date: Jul. 14, 2005

(54) Method for the immobilization of oligonucleotides

(76) Inventors: Christopher P. Adams, (US); Joseph D. Kittle JR., (US)

Correspondence Address: LOCKE LIDDELL & SAPP LLP, 600 TRAVIS, 3400 CHASE TOWER, HOUSTON, TX, 77002-3095, US

(21) Appl. No.: 11/054,650

(22) Filed: Feb. 9, 2005

Related U.S. Application Data
(62) Division of application No. 09/857,378, filed on Nov. 15, 2001, now abandoned.
(60) Provisional application No. 60/110,891, filed on Dec. 4, 1998.

(51) Int. Cl. A61K 48/00; C12Q 1/68; C07H 21/04; C12N 15/87

(52) U.S. Cl. 514/44; 435/6; 435/459; 525/54.2; 536/23.2

(57) ABSTRACT

A substantially water-soluble polymer comprises a homopolymer or a copolymer and a first subunit precursor. The first subunit precursor comprises a first nucleic acid and an ethylene-containing moiety. The first subunit precursor is either covalently or non-covalently linked to the homopolymer or copolymer. The ethylene group is preferably tethered to the 3′- or 5′-hydroxyl position of the first nucleic acid. The substantially water-soluble polymer may further comprise a tissue-specific targeting moiety and/or a bioactive compound encapsulated by the homopolymer or copolymer.

### CROSS-REFERENCES TO RELATED APPLICATIONS

This application claims priority to U.S. Provisional Application Ser. No. 60/110,891, filed on Dec. 4, 1998. The disclosure of the Provisional Application is incorporated herein by reference in its entirety for all purposes.

### BACKGROUND OF THE INVENTION

A perennial goal in the pharmacological arts is the development of methods and compositions to facilitate the specific delivery of therapeutic and other agents to cells and tissues that would benefit from such treatment, and the avoidance of the general physiological effects of the inappropriate delivery of such agents to other cells or tissues of the body. Recently, the advent of recombinant DNA technology and genetic engineering has provided the pharmacological arts with a wide spectrum of new agents that are functional genes carried in recombinant expression constructs capable of mediating expression of these genes in host cells. These developments have carried the promise of “molecular medicine,” specifically gene therapy, whereby a defective gene is replaced by an exogenous copy of its cognate, functional gene, thereby alleviating a variety of genetic diseases.

An ever-expanding array of genes, the abnormal expression of which, is associated with life-threatening human diseases is being cloned and identified. The ability to express such cloned genes in humans will ultimately permit the prevention and/or cure of many important human diseases, which now are either poorly treated or are untreatable by available therapies. As an example, in vivo expression of cholesterol-regulating genes, genes which selectively block the replication of HIV, or tumor-suppressing genes in human patients should dramatically improve treatment of heart disease, HIV, and cancer, respectively. However, currently available gene delivery strategies have been unable to produce a high level of generalized transgene expression in vivo after systemic administration to a mammalian host. This inability has precluded the development of effective gene therapy for most human diseases.

The various approaches to gene therapy include both different goals and different means of achieving those goals. The goals include gene replacement, gene correction and gene augmentation. In gene replacement, a mutant gene sequence is specifically removed from the genome and replaced with a normal, functional gene. In gene correction, a mutant gene sequence is corrected without any additional changes in the target genome. In gene augmentation, the expression of mutant genes in defective cells is modified by introducing foreign normal genetic sequences.

The means to reach the above goals include “ex vivo” transfection of a target cell, followed by introduction of the transformed cells into a suitable organ in the host mammal. Ex vivo techniques include transfection of cells in vitro with either naked DNA or DNA encapsulated in, for example, liposomes, followed by introduction into a host organ (“ex vivo” gene therapy).

There are several drawbacks to ex vivo therapy. For example, if only differentiated, replicating cells are infected, the newly introduced gene function will be lost as those cells mature and die. Ex vivo approaches can be used to transfect only a limited number of cells and cannot be used to transfect cells which are not first removed from the body. The above methods involve integration of new genetic material into the cell genome and thus constitute permanent changes to the host genome. However, some gene augmentation can be achieved using methods that do not involve changes to the genome, but which introduce DNA into a host cell where it is maintained primarily in an extrachromosomal or episomal form.

The greatest drawback to the achievement of effective gene therapy has been the inability in the art to introduce recombinant expression constructs encoding functional eukaryotic genes into cells and tissues in vivo. While it has been recognized as desirable to increase the efficiency and specificity of administration of gene therapy agents to the cells of the relevant tissues, the goal of specific delivery has not yet been achieved.

In methods other than ex vivo methods, genetic material is transferred into target cells without the use of vectors or carriers. For example, genetic material is introduced systemically through an intravenous or intraperitoneal. injection for in vivo applications, or it is introduced to the site of action by direct injection into that area. For example, it has long been recognized that DNA, by itself, injected into various tissues, will enter cells and produce a protein eliciting an immune response. See, e.g., Atanasiu et al., Academie des Sciences (Paris) 254: 4228-30 (1962); Israel et al., J. Virol. 29: 990-96 (1979); Will et al., Nature, 299: 740-42 (1982); Robinson, WO 86/00930, published 13 Feb. 1986; Felgner et al., WO 90/11092, published 4 Oct. 1990; and Debs et al., WO 93/24640, published 9 Dec. 1993. DNA by itself, however, is hydrophilic and the hydrophobic character of the cellular membrane poses a significant barrier to the transfer of naked DNA across it. Accordingly, it is generally prefer-red to use facilitators that enhance the transfer of DNA into cells on direct injection.

Facilitators that have been used are generally polycationic in nature. For example, polylysine has been widely investigated as a facilitator (Soeda et al., Gene Ther.: 1410 (1998)). Poly-lysine is not unique in providing a polycationic framework for the entry of DNA into cells. DEAE-dextran has also been shown to be effective in promoting RNA and DNA entry into cells; see, Juliano et al., Exp. Cell. Res. 73: 3-12 (1972); and Mayhew et al., Exp. Cell. Res. 77: 409-414 (1973). More recently, a dendn'tic cascade co-polymer of ethylenediamine and methyl acrylate has been shown to be useful in providing a carrier of DNA which facilitates entry into cells; see, Haensler et al., Bioconj. Chem. 4: 372-379 (1993). An alkylated polyvinylpyridine polymer has also been used to facilitate DNA entry into cells; see, Kabanov, et al., Bioconj. Chem. 4: 448-454 (1993). None of these references suggests forming a polymer from subunits including both an ethylene moiety and a nucleic acid.

Many polycationic facilitators or carriers are used to reversibly complex a polyanionic nucleic acid by an ionic binding mechanism. For example, Hennick et al., WO 97/15680, published I May 1997, have used a synthetic transfection system comprising a water dispersible or water soluble carrier fori-ned from polyacrylate, polyacrylamide and derivatives thereof. The carriers are substituted with cationic groups, which reversibly complex a nucleic acid. The use of cationic carriers to reversibly complex therapeutic nucleic acids is limited in its scope; when uncharged nucleic acid derivatives (e.g., phosphorothioates) are utilized, an ionic bonding mechanism will not be operative. Other facilitators take advantage of the ability of nucleic acids to form hydrogen bonds. Mumper et al. (Pharm. Res. 13: 701 (1996)) have reported the use of polyvinyl derivatives (e.g., polyvinylpyrrolidone, polyvinylalcohol) as interactive polymers for controlled gene delivery to muscle. The polyvinyl derivatives bind reversibly to the nucleic acid via a hydrogen bonding mechanism. There is no suggestion in either Hennick et al or Mumper et al to form polymers with nucleic acids derivatized with ethylene-containing moieties or that a nucleic acid can be polymerized to form a conjugate to which the nucleic acid is covalently bound.

Nucleic acids that are substituted with an olefin are known in the art. For example, Nagatsuge et al., (Tetrahedron 9: 3035 (1997)) have described the synthesis of 2-aminopurine derivatives with a C⁶-substituted olefin. These agents are used to form crosslinks with a target nucleobase due to their proximity to the base in a sense-antisense duplex. There is no suggestion in this reference to incorporate the olefinic group of the olefinically derivatized base into the backbone of a polymer. Furthermore, Mosaic Technologies, Inc. has introduced ethylene-containing phosphoramidite linkers that allow an ethylene group to be tethered to a nucleic acid (Acrydite™). The ethylene-derivatized nucleic acids are generally immobilized by polymerization in an acrylamide gel. See, for example, www.mostek.com, Kenney et al., Biotechniques 25: 516 (1998). The ethylene-linked nucleic acids have not been suggested for incorporation into particles or substantially water-soluble polymers. Moreover, they have not been suggested as components of a vehicle for delivering nucleic acids to a target cell.

In spite of active research in the area of delivery vehicles for therapeutic nucleic acids in gene therapy, an agent has not been developed that allows polymeric carriers to be made from ethylene-derivatized nucleic acids. In view of the number of polymerization and addition reaction pathways available to compounds bearing the ethylene group, a set of nucleic acid-derivatized polymer backbones having a great deal of structural diversity can quickly and easily be generated from nucleic acids bearing an ethylene group. The ability to rapidly generate compounds having a broad range of structural diversity allows for delivery vehicles having improved pharmacological properties to be quickly identified from a pool of diverse structures. The compounds with improved properties are useful in methods, such as gene therapy. Quite surprisingly, the present invention provides such compounds.

### SUMMARY OF THE INVENTION

It has now been discovered that nucleic acid polymers, monomers and nucleic acid analogues can be tethered to a scaffold (e.g., a polymer, oligomer, etc.) via a species comprising an ethylene moiety (e.g., acrylic- or vinyl-containing reactive group). The ethylene group is generally tethered to the 3′- or 5′-hydroxyl of the nucleic acid or nucleic acid analogue, however, this group can be attached at any position of the nucleic acid chain and to any group on either the sugar or the base of the nucleic acid or nucleic acid analogue.

The incorporation into the nucleic acid of the ethylene derivatized group can be easily accomplished by utilizing a derivative such as a phosphoramidite to which the ethylene derivatized reactive group is tethered. Phosphoramidite and other appropriate nucleic acid chemistries are well known in the art and suitable reaction schemes will be apparent to those of skill in the art.

Thus, in a first aspect, the present invention provides a substantially water-soluble polymer including a first subunit comprising a first nucleic acid. The first subunit is incorporated into the polymer using a first subunit precursor. The first subunit precursor includes the first nucleic acid and an ethylene-containing moiety.

In a second aspect, the invention provides a polymeric particle including a first subunit. The first subunit includes a first nucleic acid. The first subunit is incorporated into the polymer using a first subunit precursor. The first subunit precursor includes an ethylene-containing moiety.

In a third aspect, the instant invention provides a pharmaceutical formulation including a pharmaceutically acceptable carrier and a substantially water-soluble polymer. The polymer includes a first subunit. The first subunit includes a first nucleic acid. The first subunit is incorporated into the polymer using a first subunit precursor including the first nucleic acid and an ethylene-containing moiety.

In a fourth aspect, the present invention provides a pharmaceutical formulation including a pharmaceutically acceptable carrier and a polymeric particle.

The particle includes a first subunit. The first subunit includes a first nucleic acid. The first subunit is incorporated into the polymer using a first subunit precursor, which includes the first nucleic acid and an ethylene-containing moiety.

In a fifth aspect, the present invention provides a method for treating or preventing a condition in a subject. The method includes administering to the subject a substantially water-soluble polymer in an amount effective to treat or prevent the condition. The polymer comprises a first subunit, which includes a first nucleic acid.

The first subunit is incorporated into the polymer using a first subunit precursor, which includes the first nucleic acid and an ethylene-containing moiety.

In a further aspect, the present invention provides a method for treating or preventing a condition in a subject. The method includes administering to the subject a polymeric particle in an amount effective to treat or prevent the condition. The particle includes a first subunit, which includes a first nucleic acid. The first subunit is incorporated into the polymer using a first subunit precursor. The precursor includes the first nucleic acid and an ethylene-containing moiety.

In a still further aspect, the invention provides a method for introducing a polynucleotide into a eukaryotic cell in a living animal. The method includes contacting the cell with a composition comprising a substantially water-soluble polymer, which includes a first subunit. The first subunit includes a first nucleic acid. The first subunit is incorporated into the polymer using a first subunit precursor, which includes the first nucleic acid and an ethylene-containing moiety.

In another aspect, the invention provides a method for introducing a polynucleotide into a eukaryotic cell in a living animal. The method includes contacting the cell with a composition comprising a polymeric particle, which includes a first subunit. The first subunit includes a first nucleic acid. The first subunit is incorporated into the polymer using a first subunit precursor, which includes the nucleic acid and an ethylene-containing moiety.

Additional objects and advantages of the present invention will be apparent from the detailed description that follows.

### DETAILED DESCRIPTION OF THE INVENTION AND THE PREFERRED EMBODIMENTS

A. Definitions

As used herein the term “polymer” refers to molecules having two or more subunits (e.g., dinucleotides). As used herein, the term “nucleic acid” is used interchangeably with RNA and DNA and this term can refer to monomeric, oligomeric or polymeric species of these molecules. Moreover, nucleic acid analogues are incorporated within this definition.

“Alkyl” denotes straight-chain, branched-chain, saturated and unsaturated groups.

“Substituted alkyl” refers to alkyl as just described including one or more functional groups such as lower alkyl, aryl, acyl, halogen (i.e., alkylhalos, e.g., CF₃ hydroxy, amino, alkoxy, alkylamino, acylamino, acyloxy, aryloxy, aryloxyalkyl, mercapto, both saturated and unsaturated cyclic hydrocarbons, heterocycles and the like.

These groups may be attached to any carbon of the alkyl moiety. Moreover, the substitution can be pendent from the alkyl chain or interrupt the alkyl chain.

The term “aryl” is used herein to refer to an aromatic group, which may be a single aromatic ring or multiple aromatic rings which are fused together, linked covalently, or linked to a common group such as a methylene or ethylene moiety. The common linking group may also be a carbonyl as in benzopherione. The aromatic ring(s) may include phenyl, napthyl, biphenyl, diphenylmethyl and benzophenone among others.

The term “aryl” encompasses “arylalkyl.” The term “arylalkyl” is used herein to refer to a subset of “aryl” in which the aryl group is attached to the another group of the compound by an alkyl group as defined herein.

“Substituted aryl” refers to aryl as just described including one or more functional groups such as lower alkyl, acyl, halogen, alkyllialos (e.g. CFA hydroxy, amino, alkoxy, alkylamino, acylamino, acyloxy, mercapto and both saturated and unsaturated cyclic hydrocarbons which are fused to the aromatic ring(s), linked covalently or linked to a common group such as a methylene or ethylene moiety. The linking group may also be a carbonyl such as in cyclohexyl phenyl ketone. The ten-n “substituted aryl” encompasses “substituted arylalkyl.” “Substituted arylalkyl” defines a subset of “substituted aryl” wherein the substituted aryl group is attached to another group of the compound by an alkyl group as defined herein.

“Substituted” encompasses both single and multiple substitutions, the latter including multiple substitutions by the same substituent as well as mixtures of different substituents.

B. Introduction

In accordance with the subject invention, nucleic acid constructs together with methods of preparation and use of these constructs are provided which allow for in vivo change of the genotype and/or modulation of the phenotype of cells in a plurality of tissues of a mammalian host, following introduction of the constructs into a circulating body fluid, organ or body cavity at a sufficient dose to cause transfection of tissues and/or cells contacted by the nucleic acid. The tissues which are transformed include, for example, the lungs, heart, liver, bone marrow, spleen, lymph nodes, kidneys, thymus, skeletal muscle, ovary, uterus, stomach, small intestine, colon, pancreas, and brain in normal animals, as well as metastatic tumors and intravascular tumor emboli in tumor-bearing mammals. Particular cells which are transfected include, for example, macrophages, alveolar type I and type 11 cells, hepatocytes, airway epithelial cells, vascular endothelial cells, cardiac myocytes, myeloblasts, erythroblasts, B-lymphocytes and T-lymphocytes. The circulating bodily fluid is generally blood, but intrathecal administration can also be used.

The invention provides compositions and pharmaceutical formulations including these compositions. Methods for using the compositions of the invention are also provided. Included are methods for treating or preventing a condition in a subject and methods for delivering genetic material into a cell.

1. Compositions

(a). Substantially Water-Soluble Polymers

In a first aspect, the present invention provides a substantially water-soluble polymer comprising a first subunit, which includes a first nucleic acid. The first subunit is incorporated into the polymer using a first subunit precursor, which includes the first nucleic, acid and an ethylene-containing moiety.

The polymers of the invention can have substantially any structure achievable by using subunits having a polymerizable or otherwise reactive ethylene moiety. Thus, the polymers can be homopolymers or copolymers of two or more structurally distinct subunits. The subunits themselves can be either monomeric or polymeric. Thus, it is within the scope of the present invention to construct a single polymer from two distinct polymer molecules having similar or quite different properties. Alternatively, one or more of the subunits can be a polymer to which monomeric subunits are appended. Other permutations of the conjugation of monomers and/or polymers will be apparent to those of skill in the art and are useful in preparing the compounds of the present invention.

In a preferred embodiment, the subunits of the present polymers are attached via a cleavable moiety. Many cleaveable groups are known in the art. See, for example, Jung et al., Bio Chem. Biophys. Acta, 761: 152-162 (1983); Joshi et al., J. Biol. Chem. 265: 14518-14525 (1990); Zarling et al., J. Immunol. 124: 913-920 (1980); Bouizar et A, Eur. J. Bio Chem. 155: 141-147 (1986); Park et al., J. Biol. Chem. 261: 205-210 (1986); Browning et al., J. Immunol. 143: 1859-1867 (1989). Exemplary cleaveable moieties can be cleaved using light, heat or reagents such as thiols, hydroxylamine, bases, periodate and the like.

Cleaveable groups preferred for use in the compounds of the invention include a cleaveable moiety that is a member selected from the group consisting of disulfide, ester, imide, carbonate, nitrobenzyl, phenacyl and benzoin groups.

In an exemplary embodiment, the polymer includes at least two units joined by a photocleaveable group. The polymer is delivered to a tissue, for example, a tumor, and after some duration for a desired degree of uptake or distribution to occur, a high intensity light is focused on the tissue. The light is of sufficient energy to cleave the photocleaveable bonds and disrupt the polymeric backbone, and release the nucleic acid.

Still further preferred cleavable moieties are those that undergo cleavage due to a naturally occurring biological process. Exemplary cleavable groups according to this motif include, for example, a cleavable linker that is sensitive to the slightly acidic pH of endocytotic vacuoles. In one embodiment, the polymer is delivered to a desired tissue and taken up by the cell via encapsulation in an endocytotic vacuole, where it is cleaved into smaller fragments by the acidic environment of the vacuole.

Representative groups cleaved by biological processes include, for example, disulfides, esters, phosphodiesters and combinations thereof. Disulfides are cleaved in vivo by reducing enzymes and small molecule thiol transfer reagents. Esters undergo hydrolysis and are also catalytically cleaved by esterases. Phosphodiesters are cleaved by nucleases.

A representative disulfide-containing polymer is prepared as follows. A first ethylene-containing subunit, to which a first nucleic acid is tethered, is cross-linked, via a disulfide-containing crosslinking agent, to a second ethylene-containing subunit, to which a second nucleic acid is optionally tethered. Useful crosslinking agents are those that include both a disulfide group and two or more groups reactive with the ethylene-containing moieties of the subunits. A representative agent is N,N′-bis(acryloyl)cystamine (Hansen, Anal. BioChem. 76: 37 (1976), commercially available from Sigma, St. Louis, Mo.).

A representative ester-containing polymer is prepared as follows. A first ethylene-containing subunit, to which a first nucleic acid is tethered, is cross-linked, via an ester-containing crosslinking agent, to a second ethylene-containing subunit, to which a second nucleic acid is optionally tethered. Useful crosslinking agents are those that include both an ester group and two or more groups reactive with the ethylene-containing moieties of the subunits. Representative agents include, the bis-acryloyl- and his-methacroyl-poly(ethyleneglycol) agents commercially available from Shearwater Polymers (Huntsville, Ala.).

Other ester containing crosslinking agents are easily prepared in a few steps from commercially available starting materials, such as the active ester of acrylic acid, acrylic acid-N-hydroxysucinimide. This agent is used to prepare bis-acryloyl ester derivatives of, for example, a wide variety of unsubstituted and internally substituted U.-, co-diols. Other combinations of cleavable cross-linking agents and subunits of use in preparing the polymers of the invention will be apparent to those of skill in the art.

In another preferred embodiment, the nucleic acid and the ethylene-containing moiety are joined by a linking group. Using such linking groups, the properties of the polymer can be controlled. Properties that are usefully controlled include, for example, hydrophobicity, hydrophilicity, surface-activity and the distance of the nucleic acid from the polymer backbone. For example, in a polymer including a backbone of largely aliphatic character, the nucleic acid can be attached to the polymer backbone via a poly(ethyleneglycol) to enhance the hydrophicity of the polymer or to impart additional steric freedom to the nucleic acid. Numerous other combinations of linking groups and polymer backbones are accessible to those of skill in the art.

The hydrophilicity of the polymer can be enhanced by incorporating linking groups that include polar moieties such as amines, hydroxyls and polyhydroxyls. Representative examples include, but are not limited to, polylysine, polyethylencimine, poly(ethyleneglycol) and poly(propyleneglycol). Suitable functionalization chemistries and strategies for these compounds are known in the art. See, for example, Dunn, R. L., et al., Eds. POLYMERIC DRUGS AND DRUG DELIVERY SYSTEMS, ACS Symposium Series Vol. 469, American Chemical Society, Washington, D.C. 1991.

The hydrophobicity of the polymer can be modulated by using a hydrophobic linking group such as, for example, long chain diamines, long-chain thiols, (x, o-amino acids, etc. Representative hydrophobic spacers include, but are not limited to, 1,6-hexanediamine, 1,8-octanediamine, 6-aminohexanoic acid and 8-aminooctanoic acid.

The polymer can also be made surface-active by using a linking group having surfactant properties. Compounds useful for this purpose include, for example, aminated or hydroxylated detergent molecules such as, for example, 1-aminododecanoic acid.

In another embodiment, the linking group serves to distance the nucleic acid from the polymer backbone. Groups with this characteristic have several uses. For example, a nucleic acid held too closely to the polymer backbone may not be able to hybridize with an incoming complementary nucleic acid strand, or it may hybridize unacceptably slowly. When an incoming nucleic acid strand is itself sterically demanding, the hybridization can be undesirably slowed, or not occur at all, due to the monolithic polymer backbone hindering the approach of the two complementary strands.

Thus, linking groups that provide distance between the nucleic acid and the polymer backbone can serve to enhance nucleic acid hybridization.

In a still further preferred embodiment, the linking group includes a cleavable moiety. The discussion above concerning cleavable moieties is generally applicable to this embodiment as well.

In another embodiment, the physicochemical characteristics (e.g., hydrophobicity, hydrophilicity, surface activity, conformation) of the polymer are altered by attaching a monovalent moiety which is different in composition than the constituents of the bulk polymer and which does not bear a nucleic acid. As used herein, “monovalent moiety” refers to organic molecules with only one reactive functional group. This functional group attaches the molecule to the polymer backbone. “Monovalent moieties” are to be contrasted with the bifunctional linking groups described above. Such monovalent groups are used to modify the hydrophilicity, hydrophobicity, binding characteristics, etc. of the polymer. Examples of groups useful for this purpose include long chain alcohols, amines, fatty acids, fatty acid derivatives, poly(ethyleneglycol) monomethyl ethers, etc.

In the polymers of the invention, the ethylene-containing moiety can have substantially any structure deemed useful for a particular application. Thus, the ethylene-containing moiety can be, for example, a saturated alkyl group an unsaturated alkyl group, a carbohydrate, an amino acid or peptide, a polyether, a polyamine, etc.

In a presently preferred embodiment, the ethylene-containing moiety includes a member selected from CH₂═CHX¹, CH₂═CX²Y¹ and combinations thereof.

In these ethylene-containing moieties X¹, X² and Y¹ are members independently selected from H, (═O), NR′R², OH, and OR³, R¹, R² and R³ are members independently selected from H, alkyl, substituted alkyl, aryl and substituted aryl.

In a still further preferred embodiment, R¹, R² and R³ independently selected from H, alkyl and substituted alkyl, more preferably from H, alkyl and alkyl substituted with at least one moiety selected from OH, 0 and combinations thereof.

In yet another preferred embodiment, at least one of R¹, R² and R³ includes a moiety selected from poly(ethyleneglycol), poly(propyleneglycol) and combinations thereof.

In another preferred embodiment, the polymer backbone includes a group that is derived from a member selected from acrylate, acrylamide, C₁-C₆ alkylacrylate, (alkyl)acrylamide, methylmethacrylate, triethyleneglycolmethacrylate, poly(ethyleneglycol)metliacrylate, hydroxyethylmethacrylate, glycerylmethacrylate, vinyl alcohol, ethylcyanoacrylate and combinations thereof.

(i). Framework Components

In another preferred embodiment, the monomeric or polymeric ethylene-containing subunits are attached to a secondary polymeric component that is derivatized to allow for such attachment. In this embodiment, the polymeric moiety to which the subunits are attached is referred to as a “framework component.” Examples of framework components suitable for use in the methods of the present invention include, but are not limited to, polymers, liposomes, micelles, colloids, biological particles and non-biological particles (e.g., silica beads, polymeric beads, gels, etc.). These various types of framework components are discussed briefly below and in more detail further below under the description of covalent and noncovalent frameworks. The detailed descriptions of each of these framework components are provided under the headings of covalent and noncovalent framework components only for ease of discussion and should not be construed as limiting the scope of useful framework structures. It is understood that the present invention is intended to encompass all types of frameworks capable of presenting functional groups that are reactive towards the ethylene-containing moiety of the subunits.

As noted above, examples of framework components suitable for use in the methods of the present invention include, but are not limited to, polymers, liposomes, micelles, colloids, dendrimers and biological particles. The terms “polymer” and 11 polymeric” are art-recognized terms and, as used herein, include reference to a structural framework including repeating monomer units. The ternis also include reference to homopolymers and copolymers. Linear polymers, branched polymers and cross-linked polymers are also encompassed within the terms “polymer” and “polymeric.”

The terms “liposome,” “micelles,” and “colloids” are art-recognized terms and, as used herein, these terms also include the derivatized versions, e.g., liposome derivatives, cross-linked liposomes, etc.

The term “biological particle” includes reference to both covalent molecules, e.g., sugars, proteins, lipid, small molecules, protein aggregates, and nucleic acids, and noncovalent particles, e.g., modified cells (e.g., which have been derivatized, modified chemically or transfected with an exogenous nucleic acid), or modified viruses, e.g. viral particles. The use of “biological particles” as framework components is distinguished from such particles as they occur in their natural state because the subject framework components are typically modified to present a nucleic acid.

In each of the embodiments discussed hereinbelow, it is generally understood that prior to their being reacted with the ethylene-containing nucleic acid conjugate, the covalent framework component is either “naturally” or “synthetically primed” for reaction with the ethylene group of the conjugate. “Naturally primed” polymers are those that can be coupled to the ethylene-containing moiety without prior derivitization of the polymers. “Synthetically primed” polymers are those that undergo additional modification prior to conjugation with the ethylene group.

In an exemplary embodiment, a framework component that bears amines on its surface is reacted with an activated ester (e.g., N-hydroxysuccinimide) of a compound, such as acrylic acid, thereby converting the amines to an ethylene-containing group that can be reacted with the ethylene-containing subunits discussed above. In another embodiment, the surface of the amine-containing polymer is utilized in an addition reaction, such as the Michael addition, in which case the amine-containing polymer is naturally primed for reaction with the ethylene-containing moiety of the conjugate. Many other priming strategies and reaction sequences can be utilized to prepare an appropriately reactive framework component. Such reaction sequences are well known, and easily accessible, to those of skill in the art.

Covalent Framework Components

In one embodiment, the monomeric units of a framework component are joined covalently. Exemplary covalent frameworks include, but are not limited to, cross-linked liposomes, biological particles (e.g., sugars, proteins, peptides, lipids, or small molecules) and polymeric materials (see, e.g., Siraganian et al., ImmunoChem. 12: 149-155 (1975); Wofsy et al., J. Immunol. 121: 593-601 (1978); Barlocco et al., Farnlac. 48: 387-96 (1993); Castagnino et al., Jpn. Heart J. 31: 845-55 (1990); Costa et al., BioChem. Pharmacol. 34: 25-30 (1985); Dembo et al., J. Immunol. 122: 518-28 (1979); Holliger et al., Proc. Natl. Acad. Sci. U.S.A. 90: 6444-8 (1993); Piergentili et al., Farmaco 49: 83-7 (1994); Portoghese et al., J. Med Chem. 34: 1292-6 (1991); Kizuka et al.,. J. Am. Chem. Soc. 30: 722-6 (1987)).

In certain embodiments, proteins, e.g., albumin, can be used as a framework component for presenting large numbers of groups (Roy et al., Can. J. Chem. 68: 2045-2054 (1990)), thereby mimicking natural glycoprotein inhibitors.

In presently preferred embodiments, polymers are used as the framework component. Polymers are a versatile framework system (see, e.g., Spaltenstein et al., J. Am. Chem. Soc. 113: 686 (1991); Mammen et al., J. Med. Chem. 38: 4179 (1995)). In a preferred embodiment, the ethylene-containing nucleic acids of the present invention are attached to a framework component comprising a polymeric backbone through a linker group.

Polymers can be purchased from commercial sources or, alternatively, they can be prepared using methods known to those of skill in the art (See, e.g., Sandler, et al., POLYMER SYNTHESES; Harcourt, Brace: Boston, 1994; Shalaby et al., J Polymers of Biological and Biomedical Significance (A CS Symposium Series 540; American Chemical Society: Washington, D.C., 1994). Moreover, polymeric frameworks are easily, rapidly and convergently synthesized (see, e.g., Spaltenstein et al., J. Am. Chem. Soc. 113: 686 (1991); Mammen et al., J. Med. Chem. 38: 4179 (1995)).

Moreover, polymers provide a number of advantages as the framework component, because the characteristics of the polymer can be varied, modulated and controlled as desired. For instance, characteristics which can be varied and controlled include, but are not limited to, conformal flexibility; solubility; hydrophilicity; modulation of conformation and flexibility in solution through variations in temperature and ionic strength, etc. As such, the use of polymers readily allows for the modulation of various physical properties of the framework. Additionally, the characteristics of the polymers can be designed to vary the flexibility of the polymer, the distance between the functional groups (e.g., bioactive sidechains), the length of the spacer group or linker between the polymer backbone and the functional groups, etc.

The chemistry of high molecular weight polymers is a well-developed science, and organic polymers provide a very important class of compounds to use for nucleic acid conjugates. Such compounds have high molecular weights, can present very large numbers of copies of the nucleic acid and can present more than one nucleic acid simultaneously.

In preferred embodiments, modified polymeric materials for use in the present invention have low antigenicity and low toxicity. In other preferred embodiments, polymeric frameworks are selected to be compatible with water, to have various molecular weights and to be capable of having a range of different groups attached to the polymer backbone. Polymer backbones of the present invention can also be selected for ease of synthesis.

Intrinsically biocompatible polymers containing functional groups appropriate for the addition of sidechains are preferred (Shalaby, et al., J. Polymers of Biological and Biomedical Significance (ACS Symposium Series 540); American Chemical Society: Washington, D.C., 1994). Exemplary polymers include, but are not limited to, polyethylene oxide or polyethyleneglycol (Harris, J. M., POLY(ETHYLENE GLYCOL) CHEMISTRY: BIOTECHNICAL AND BIOMEDICAL APPLICATIONS; Plenum: New York, 1992; Horton, D., ADVANCES IN CARBOHYDRATE CHEMISTRY AND BIOCHEMISTRY; Academic Press: San Diego, 1995) as well as derivatives of acrylamide and N-vinylpyrollidone, linked oligomers of oligoethylene glycol, linked oligomers of dextran and others.

Other preferred framework components for use in the methods of the present invention have demonstrated utility, for example, as plasma extenders, drug excipients or binders, food additives or as inert or erodible materials used in vivo. For example, poly(ethylene glycol), poly(lactic acid), poly(glycolic acid) and poly(vinyl pyrrolidone) can be used within the framework component in the methods of the present invention.

Preferred polymers contain reactive groups, such as amines, carboxylic acids, etc. A number of synthetic and naturally-occurring polymers containing carboxylic acid functionality (or capable of being suitably modified) have been previously used in vivo. Such polymers are capable of derivatization to facilitate linkage of the nucleic acid-containing subunit groups, described herein. Polymers containing internally cyclized carboxylic acid functionality, such as anhydride or succinimide groups, are also preferred.

Other preferred polymers include, but are not limited to, derivatives of maleic anhydride and malic acid. Exemplary copolymers include, but are not limited to, styrene-maleic anhydride and alpha-olefin-maleic acid copolymers (such as divinylether-maleic acid). In other embodiments, sodium carboxymethyl cellulose, chondroitin sulfate and poly(methacrylate/acrylate) materials can be used. In still other embodiments, polymers without activated carboxylic acids can be used, such as dextran sulfate.

Other exemplary polymeric framework components include, but are not limited to, poly(ester), poly(anhydride), poly(carbohydrate), polyols, poly(acrylate), poly(methaerylate), poly(ether) and poly(amino acid). Still other exemplary polymeric frameworks include, but are not limited to, polyamines, poly(glutamic acid), poly(aspartic acid), dextran, dextran sulfate, poly(maleic anhydride-co-vinyl ether), poly(succinimide), poly(acrylic anhydride), poly(ethylene glycol), poly(lactic acid), poly(glycolic acid), poly(amine), poly(vinyl pyrrolidone), poly(styrene-maleic anhydride), alpha-maleic acid, hyalouronic acid, sodium carboxymethyl cellulose, chondroitin sulfate, poly(acrylate), poly(acrylamide), poly(glycerol) and starch. Table I sets forth a list of representative polymers useful in practicing the present invention.

Table 1. Exemplary polymers Poly(ethylene glycols) Poly(ethylene-vinyl acetate) Poly(amides) Poly(acrylamides) poly(peptides) Poly(urethanes) Poly(aminoacids) Poly(methacrylates) poly(aspartic acid) Poly(acrylates) Poly(glutamic acid) Poly(maleic acid poly(lysine), others copolymers) proteins(gelatins) Poly(anhydrides) Poly(esters) Poly(orthoester) poly(lactic acid), polylactide poly(glycolide) poly(caprolactone) poly(tartrate) Polysaccharides cellulose alginates starch Dextran derivatives Poly(N-vinylpyrrolidone) It will be appreciated by the skilled artisan that substantially any polymeric material which is capable of presenting a plurality of functional groups is suitable for use ZD in the present invention. Polymers can be modified, e.g., as described above or by derivatization, e.g., with bifunctional cross-linking reagents, to provide functionalities suitable for presenting functional groups as described in more detail below.

Noncovalent Framework Components

A plurality of ethylene-containing nucleic acids can also be joined to a non-covalent framework. Exemplary noncovalent frameworks include, but are not limited to, liposomes, micelles, colloids, protein aggregates, modified cells, and modified viral particles. For example, functional and/or ancillary groups can be tethered to the head groups of molecules in liposomes, membranes or surfaces (see, e.g., Kingery-Wood et al., J. Am. Chem. Soc. 114: 7303-7305 (1992); Spevak et al., J. Am. Chem. Soc. 115: 1146-1147 (1993); Spevak et al., J. Med Chem. 39: 1018-1020 (1996)).

Liposomes and micelles are art-recognized terms and include macroscopic particles made up of aggregates of surfactants. In one embodiment, the compound of the invention can present groups on a liposome or micelle (Spevak et al., J. Am. Chem. Soc. 115: 1146-1147 (1993); Charych et al., Chem. & Biol. 3: 113-120 (1996)). This system closely mimics the shape of the target cell, and can be designed to present a surface that closely matches that of the target cell both in terms of group type and group density. For example, lipid molecules containing functional groups (e.g., neuraminic acid (NeuAc)) as polar head groups can be reconstituted into liposomes. Liposomes have favorable bio-compatibility and are fairly easy to synthesize.

In other embodiments, biological particles including, for example, modified cells or modified viruses can be used as the framework component for presentation of the functional group. Thus, proteins, peptides, polysaccharides, fragments of cell membranes, or modified intact cells (e.g., erythrocytes), modified bacterial cells or modified viruses can be used as the framework component in certain embodiments.

Activated Framework Component

As used herein, the term “activated framework component” refers to a framework component, as described above, containing groups that can be activated, by means of an “activating group,” and subsequently reacted with at least one functional group, ancillary group and/or spacer group that includes a group that is reactive with the ethylene-derivatized nucleic acid. Appropriate functionality includes, for example, carboxyl (acid form and salts), hydroxyl, sulfhydryl, amide, carbamate, amino, ketone, aldehyde, olefin, aromatic, etc. The polymers can be activated prior to exposing them to the functional group (“preactivation”), or can be activated in the presence of the functional group (“in situ”).

The activation step can entail derivatizing the polymer with groups capable of undergoing reactions with nucleophiles or electrophiles (e.g., forming active esters, halo derivatives, etc.). Further, it is within the scope of the present invention to activate polymers such that they are able to participate in dipolar additions (e.g., 1,3- and 1,4-dipolar addition), cycloaddition reactions (e.g., Diels-Alder type reactions) and polymerization reactions by cationic, anionic or radical intitiated mechanisms.

Carboxyl groups can be activated for reaction with nucleophiles by the use of, for example, cyclic or linear anhydrides, activated esters (e.g., N-hydroxysuccinimide, nitrophenol, 4-hydroxy-3-nitrobenzene sulfonic acid, etc.), acid chlorides, imidazolides (e.g., from carbonyldiimidazole), carboxylic acid and esters. Carboxylic acid containing polymers may also be activated by forming adducts between the carboxyl group and agents such as, dicyclohexylcarbodiimide, 1-(3-dimethylaminopropyl)-3-ethylearbodiimide, alkyl chloroformates, chlorosilanes, pyridiniurn salts BU₃N, etc.

The selection of appropriate activating groups for the carboxyl functionality will be apparent to those of skill in the art. It will be similarly apparent to those of skill in the art which reaction systems will be amenable to, or will require, in situ activation or preactivation.

Hydroxyl groups can be activated by the use of carbonates formed by reaction with, for example, alkyl or aryl haloformates (e.g., vinylchloroformate, i-butylchloroformate, p-nitrophenylehlorofonnate, etc.), cyanogen bromide or phosgene. In aspects utilizing polymers containing vic-diol groups (e.g., dextran and other polysaccharides) oxidation using peniodate compounds can be used to provide reactive carbonyl moieties on the polymeric backbone. The carbonyl moieties are then reacted with activating groups, such as vinylamine derivatives (e.g., 4-aminostyrene, etc.).

In a preferred embodiment, a hydroxyl-containing polymer is reacted with an active ester of acrylic acid, such as the N-hydroxysuccinimide ester to form a cleavable ester linkage between the polymer framework and the nucleic acid. (In another embodiment, the hydroxyl groups are reacted with an agent, such as vinyl bromide, vinyl benzyl choloride, etc.). Additional methods of activating polymers bearing hydroxyl groups will be apparent to those of skill in the art.

Polymers bearing sulfhydryl groups can be activated using dithiobispyridyl compounds such as, for example, 2,2′-dithiobis(5-nitropyridine), 2,2′-dithiobis(pyridine), etc. Additional methods of use in activating sulfhydryl-bearing polymers will be apparent to those of skill in the art.

It will be appreciated by those of skill in the art that the above activation reactions are set forth as examples only and that many further alternatives to these schemes exist.

In preferred embodiments, the polymeric framework is polycationic. Polycationic groups have been shown to facilitate the entry of polyanionic nucleic acids into cell. Useful polycationic motifs include polyamines such as polylysine.

Additional polymers, linker groups functional groups and combinations thereof within both the scope and spirit of the present invention will be apparent to those of skill in the art.

(ii). Nucleic Acids

Any nucleic acid can be used to construct the compositions of the invention. Particularly preferred naturally occurring nucleic acid molecules include genomic deoxyribonucleic acid (DNA) and genomic ribonucleic acid (RNA), as well as the several different forms of the latter, e.g., messenger RNA (mRNA), transfer RNA (tRNA), and ribosomal RNA (rRNA). Also included are the different DNAs which are complementary (cDNA) to the different RNAs. Synthetic DNA or a hybrid thereof with naturally occurring DNA, is also encompassed within the scope of the instant disclosure.

The nucleic acid compositions used in the present invention may be either single-stranded or double-stranded, may be linear or circular, e.g., a plasmid, and are either oligo- or poly-nucleotides. They may comprise a single base or base pair, or may include as many as 20 thousand bases or base pairs (20 kb), or more.

In addition to these naturally occurring materials, the nucleic acid compositions used in the present invention can also include synthetic compositions, e.g., nucleic acid analogs, synthetic nucleic acids. These have been found to be particularly useful in antisense methodology, which is the complementary hybridization of relatively short oligonucleotides to single-stranded RNA or single-stranded DNA, such that the normal, essential functions of these intracellular nucleic acids are disrupted. See, e.g., Cohen, OLIGONUCLEOTIDES: ANTISENSE INHIBITORS OF GENE EXPRESSION, CRC Press, Inc., Boca Raton, Fla. (1989).

The nucleic acid composition to be transferred to a target cell in accordance with the present invention preferably has an appropriate open reading frame and promoter to express a protein, as well as any other regulatory sequences which may be appropriate to expression. Nucleic acid compositions to be delivered by means of the methods of the present invention can be designed and constructed so as to be appropriate for the particular application desired, all of which is well within the ordinary skill of the artisan in this field.

The nucleic acid molecules which are delivered to cells using the multifunctional molecular complex and methods of the present invention may, for example, serve as: 1) genetic templates for proteins that function as prophylactic and/or therapeutic immunizing agents; 2) replacement copies of defective, missing or non-functioning genes; 3) genetic templates for therapeutic proteins; 4) genetic templates for antisense molecules and as antisense molecules per se; or 5) genetic templates for ribozymes.

In the case of nucleic acid molecules which encode proteins, the nucleic acid molecules preferably comprise the necessary regulatory sequences for transcription and translation in the target cells of the individual animal to which they are delivered.

In the case of nucleic acid molecules which serve as templates for antisense molecules and ribozymes, such nucleic acid molecules are preferably linked to regulatory elements necessary for production of sufficient copies of the antisense and ribozyme molecules encoded thereby respectively.

The nucleic acid molecules are free from retroviral particles and are preferably provided as DNA in the form of plasmids.

In some cases, it may be desirable to use constructs that produce long term transgene effects in vivo, either by integration of the transgene into host cell genomic DNA at high levels or by persistence of the transgene in the nucleus of cells in vivo in stable, episomal form. Integration of the transgene into genomic DNA of host cells in vivo may be facilitated by administering the transgene in a linearized form (either the coding region alone, or the coding region together with 5′ and 3′ regulatory sequences, but without any plasmid sequences present). It is possible to further increase the incidence of transgene integration into genomic DNA by incorporating a purified retroviral enzyme, such as the HIV-1 integrase enzyme, into the lipid carrier-DNA complex. Appropriate flanking sequences are placed at the 5′ and 3′ ends of the transgene DNA. These flanking sequences have been shown to mediate integration of the HIV-1 DNA into host cell genomic DNA in the presence of HIV-I integrase. Alternatively, duration of transgene expression in vivo can be prolonged by the use of constructs that contain non-transforming sequences of a virus such as Epstein-Barr virus, and sequences such as oriP and EBNA-1, which appear to be sufficient to allow heterologous DNA to be replicated as a plasmid in mammalian cells (Buhans et al., Cell 62: 955 (1986)).

The nucleic acid constructs for use in the invention include several forms, depending upon the intended use of the construct. Thus, the constructs include, for example, vectors, transcriptional cassettes, expression cassettes and plasmids. The transcriptional and translational initiation region (also sometimes referred to as a “promoter,”), preferably comprises a transcriptional initiation regulatory region and a translational initiation regulatory region of untranslated 5′ sequences, “ribosome binding sites,” responsible for binding mRNA to ribosomes and translational initiation. it is preferred that all of the transcriptional and translational functional elements of the initiation control region are derived from or obtainable from the same gene. In some embodiments, the promoter will be modified by the addition of sequences, such as enhancers, or deletions of nonessential and/or undesired sequences. “Obtainable,” as used herein, refers to a promoter having a DNA sequence sufficiently similar to that of a native promoter to provide for the desired specificity of transcription of a DNA sequence of interest. It includes natural and synthetic sequences as well as sequences which may be a combination of synthetic and natural sequences.

For the transcriptional initiation region, or promoter element, any region may be used with the proviso that it provides the desired level of transcription of the DNA sequence of interest. The transcriptional initiation region may be native to or homologous to the host cell, and/or to the DNA sequence to be transcribed, or foreign or heterologous to the host cell and/or the DNA sequence to be transcribed. “Foreign to the host cell,” as used herein, refers to sequences in which the transcriptional initiation region is not found in the host into which the construct comprising the transcriptional initiation region is to be inserted. “Foreign to the DNA sequence,” as used herein, refers to a sequence in which a transcriptional initiation region that is not normally associated with the DNA sequence of interest. Efficient promoter elements for transcription initiation include, for example, the SV40 (simian virus 40) early promoter, the RSV (Rous sarcoma virus) promoter, the Adenovirus major late promoter, and the human CMV (cytomegalovirus) immediate early I promoter.

Inducible promoters also find use with the subject invention where it is desired to control the timing of transcription. Examples of promoters include but are not limited to those obtained from a beta-interferon gene, a heat shock gene, a metallothionein gene or those obtained from steroid hormone-responsive genes, including insect genes such as that encoding the ecdysone receptor. Such inducible promoters can be used to regulate transcription of the transgene by the use of external stimuli such as interferon or glucocorticoids. Since the arrangement of eukaryotic promoter elements is highly flexible, combinations of constitutive and inducible elements also can be used. Tandem arrays of two or more inducible promoter elements may increase the level of induction above baseline levels of transcription which can be achieved when compared to the level of induction above baseline which can be achieved with a single inducible element.

Generally, the regulatory sequence comprises DNA up to about 1.5 Kb 5′ of the transcriptional start of a gene, but this sequence can be significantly smaller. The regulatory sequence can be modified at a position corresponding to the first codon of the desired protein by site-directed mutagenesis (Kunkel, Proc. Natl. Acad. Sci. (USA) 82: 488-492 (1985)) or by introduction of a convenient linker oligonucleotide by ligation, if a suitable restriction site is found near the N-terminal codon. In a preferred embodiment, a coding sequence with a compatible restriction site may be ligated at the position corresponding to codon #1 of the gene. This substitution may be inserted in such a way that it completely replaces the native coding sequence and thus the substituted sequence is flanked at its 3′ end by the gene terminator and polyadenylation signal.

Transcriptional enhancer elements optionally are included in the expression cassette. “Transcriptional enhancer elements,” as used herein, refer to DNA sequences which are primary regulators of transcriptional activity and which can act to increase transcription from a promoter element. These elements generally do not have to be in the 5′ orientation with respect to the promoter in order to enhance transcriptional activity. The combination of promoter and enhancer element(s) used in a particular expression cassette can be selected by one skilled in the art to maximize specific effects. Different enhancer elements can be used to produce a desired level of transgene expression in a wide variety of tissue and cell types. For example, the human CMV immediate early promoter-enhancer element can be used to produce high-level transgene expression in many different tissues in vivo.

Examples of other enhancer elements which confer a high level of transcription on linked genes in a number of different cell types from many species include enhancers from SV40 and RSV-LTR. The SV40 and RSV-LTR are essentially constitutive. They may be combined with other enhancers which have specific effects, or the specific enhancers may be used alone. Thus, where specific control of transcription is desired, efficient enhancer elements that are active only in a tissue-, developmental-, or cell-specific fashion include immunoglobulin, interleukin-2 (IL-2) and beta-globin enhancers are of interest. Tissue-, developmental-, or cell-specific enhancers can be used to obtain transgene expression in particular cell types, such as B-lymphocytes and T-lymphocytes, as well as myeloid, or erythroid progenitor cells. Alternatively, a tissue-specific promoter such as that derived from the human cystic fibrosis transmembrane conductance regulator (CFTR) gene can be fused to a very active, heterologous enhancer element, such as the SV40 enhancer, in order to confer both a high level of transcription and tissue-specific transgene transcription. In addition, the use of tissue-specific promoters, such as LCK, may allow targeting of transgene transcription to T lymphocytes. Tissue specific transcription of the transgene may be important, particularly in cases where the results of transcription of the transgene in tissues other than the target tissue would be deleterious.

Tandem repeats of two or more enhancer elements or combinations of enhancer elements may significantly increase transgene expression when compared to the use of a single copy of an enhancer element; hence enhancer elements find use in the expression cassette. The use of two different enhancer elements from the same or different sources flanking or within a single promoter can in some cases produce transgene expression in each tissue in which each individual enhancer acting alone would have an effect, thereby increasing the number of tissues in which transcription is obtained. In other cases, the presence of two different enhancer elements results in silencing of the enhancer effects. Evaluation of particular combinations of enhancer elements for a particular desired effect or tissue of expression is within the level of skill in the art.

Although generally it is not necessary to include an intron in the expression cassette, an intron comprising a 5′ splice site (donor site) and a 3′ splice site (acceptor site) separated by a sufficient intervening sequence to produce high level, extended in vivo expression of a transgene administered iv or ip can optionally be included. Generally, an intervening sequence of about 100 bp produces the desired expression pattern and/or level, but the size of the sequence can be varied as needed to achieve a desired result. The optional intron placed 5′ to the coding sequence results in high level extended in vivo expression of a transgene administered iv or ip but generally is not necessary to obtain expression. Optimally, the 5′ intron specifically lacks cryptic splice sites which result in aberrantly spliced mRNA sequences. If used, the intron splice donor and splice acceptor sites, arranged from 5′ to 3′ respectively, are placed between the transcription initiation site and the translational start codon.

Alternatively, the intervening sequence may be placed 3′ to the translational stop codon and the transcriptional terminator or inside the coding region. The intron can be a hybrid intron with an intervening sequence or an intron taken from a genomic coding sequence. An intron 3′ to the coding region, particularly one of less than 100 bp, or any intron which contains cryptic splice sites may under certain condition substantially reduce the level of transgene expression produced in vivo. A high level of in vivo expression of a transgene can also be achieved using a vector that lacks an intron. Such vectors therefore are of particular interest for in vivo transfection.

Downstream from and under control of the transcriptional initiation regulatory regions is, preferably, a multiple cloning site for insertion of a nucleic acids sequence of interest which will provide for one or more alterations of host genotype and modulation of host phenotype. Conveniently, the multiple cloning site may be employed for a variety of nucleic acid sequences in an efficient manner. The nucleic acid sequence inserted in the cloning site may have any op en reading frame encoding a polypeptide of interest, for example, an enzyme, with the proviso that where the coding sequence encodes a polypeptide of interest, it should lack cryptic splice sites which can block production of appropriate mRNA molecules and/or produce aberrantly spliced or abnormal mRNA molecules. The nucleic acid sequence may be DNA; it also may be a sequence complementary to a genomic sequence, where the genomic sequence may be one or more of an open reading frame, an intron, a non-coding leader sequence, or any other sequence where the complementary sequence will inhibit transcription, messenger RNA processing, for example splicing, or translation.

A number of nucleic acid sequences are of interest for use in in vivo gene therapy. Where the nucleic acid codes for a polypeptide, the polypeptide may be one which is active intracellularly, a transmembrane protein, or it may be a secreted protein. It may also code for a mutant protein, for example, which is normally secreted, but which has been altered act intracellularly. The nucleic acid may also be a DNA sequences coding for mRNA (antisense or ribozyme sequences such as those to HIV-REV or BCR-ABL sequences) or for proteins such as transdominant negative mutants, which specifically prevent the integration of HIV genes into the host cell genomic DNA, replication of HIV sequences, translation of HIV proteins, processing of HIV mRNA or virus packaging in human cells; the LDL (low density lipoprotein) receptor, which specifically lowers serum cholesterol; and proteins such as granulocyte macrophage colony stimulating factor (GM-CSF) which can stimulate the production of white blood cells from the bone marrow of immunocompromised patients and produce significant anti-tumor activity or cystic fibrosis transmembrane conductance regulator (CFTR) for the treatment of cystic fibrosis. These and other beneficial (therapeutic) nucleic acid sequences can be expressed in appropriate cells in vivo using this invention.

Examples of beneficial therapeutic nucleic acid sequences are those encoding molecules having superoxide dismutase activity or catalase activity to protect the lung from oxidant injury; endothelial prostaglandin synthase to produce prostacyclin and prostaglandin E2; and antiprotease alpha-I antitrypsin. Thus, this approach could dramatically improve the treatment of acquired immune deficiency syndrome (AIDS), cystic fibrosis, cancer, heart disease, autoimmune diseases and a variety of life threatening infections. For the treatment of AIDS, anti-TAT, REV, TAR or other critical anti-HIV sequences can be used, particularly for expression of the appropriate coding sequences in T lymphocytes, macrophages and monocytes which can be achieved following iv administration of the appropriate coding sequences; expression of wild-type CFTR gene in the lungs of cystic fibrosis patients (see, Collins, Science 256: 774-783 (1992)); antisense sequences to over-expressed, transforming oncogenes, such as myc or ras in tumors; and genes which block activity of activated T cell clones which attack myelin in multiple sclerosis or other targets in autoimmune diseases. A T-cell lymphocyte clone activated to recognize and attack myelin can be targeted by using an anti-sense sequence, ribozyme sequence or transgene coding for a transdominant negative mutant which specifically blocks surface expression on the T-cell of T-cell receptor components which mediate recognition and/or attack of myelin-sheathed cells.

The choice of termination region employed will primarily be one of convenience, since termination regions appear to be relatively interchangeable. The termination region may be native to the intended nucleic acid sequence of interest, or it may be derived from another source. Convenient termination regions are available and include the 3′ end of a gene terminator and polyadenylation signal from the same gene from which the 5′ regulatory region is obtained. Adenylation residues, preferably more than 32 and up to 200 or more as necessary may be included in order to stabilize the mRNA. Alternatively, a terminator and polyadenylation signal from different gene/genes may be employed with similar results. Specific sequences which regulate post-transcriptional mRNA stability may optionally be included. For example, certain polyA sequences (Volloch et al., Cell 23: 509 (1981)) and beta-globin mRNA elements can increase mRNA stability, whereas certain AU-rich sequences in mRNA can decrease mRNA stability (Shyu et al., Genes and Devel. 3: 60 (1989)). In addition, AU regions in Ynon-coding regions may be used to destabilize mRNA if a short half-life mRNA is desirable for the gene of interest.

The construct may additionally include sequences for selection, such as a neomycin resistance gene or a dihydrofolate reductase gene and/or signal sequences to regenerate recombinant proteins that are targeted to different cellular compartments or secreted when the wild type sequence is not. Any of a variety of signal sequences may be used which are well-known to those skilled in the art. These signal sequences may allow generation of new vaccine strategies or produce soluble antagonists directed against specific cell surface receptors such as transformed oncogenes. The sequences for selection may be on a separate plasmid and cotransfected with the plasmid carrying the therapeutic nucleic acid. Where a carrier is used, the selection plasmid may be complexed to a different carrier or to the same carrier as the therapeutic plasmid.

The recombinant coding-sequence Ranked at its 5′ end by the promoter and regulatory sequences and at its 3′ end by a terminator and regulatory sequences may be introduced into a suitable cloning plasmid (e.g., pUC 18, pSP72) for use in direct DNA uptake in host cells following introduction into the host. The nucleic acid construct also may be complexed with a carrier such as lipid carriers, particularly cationic lipid carriers.

As discussed above, the nucleic acids can be either single- or double-stranded or combinations thereof. In a preferred embodiment the first nucleic acid tethered to the polymer is hybridized to a second nucleic acid. In a further preferred embodiment, the first nucleic acid is a single-stranded nucleic acid. In another preferred embodiment, the first nucleic acid is a double-stranded nucleic acid. In these embodiments, the second nucleic acid can be either single- or double stranded, forming either duplexes or triplexes with the immobilized first nucleic acid, respectively.

(b). Particles

In another aspect, the invention provides a polymeric particle including a first subunit. The first subunit includes a first nucleic acid. The first subunit is incorporated into the polymer using a first subunit precursor. The first subunit precursor includes an ethylene-containing moiety. The embodiments of this aspect are substantially similar to those discussed above in the context of the nucleic acids and substantially water-soluble polymers and many of the polymers discussed above can be used to forin the particles of the invention.

Many different types of microparticles for drug delivery are known in the art. Of particular relevance to the present invention are those microparticles that are prepared from ethylene-containing monomers, such as acrylate derivatives. Microparticles prepared from acrylic monomers have attracted a great deal of attention as colloidal drug carriers because of their case of preparation. Cyanoacrylate particles are both lysosometric, biodegradable and blocompatible (Couvrer et al., J. Pharmacol. Sci. 68: 1521 (1979)). Cyanoacrylate particles have been used in a number of different applications, such as ocular drug delivery, carriers of monoclonal antibodies and for targeting chemotherapeutic agents to cancer cells Juncel et al., J. Biomed. Mat. Res. 29: 721 (1995)).

Many methods have been developed to form discrete nanoparticles from acrylate monomers. These include, for example, aqueous anionic polymerization at a low pH (see, for example, Kreuter et al., Int. J. Pharmacol. 16: 105 (1983); Couvrer et al., J. Pharm. Pharmacol. 31: 331 (1979); Douglas et al., J. Colloid. Interface Sci. 101: 149 (1984)). Cyanoacrylate microspheres have also been prepared by dispersion polymerization in an acidic aqueous solution (Tuncel, supra; Sjöhohn et al., J. Pharm. Exp. Ther. 211: 656 (1979)). Methods have also been developed to form a population of microspheres, the members of which are of practically a single size. For example, Amsden (Pharm. Res. 16: 140 (1999)) has developed a method involving the injection of a solution to be emulsified and formed into microspheres into a stabilizing solution flowing past an injection point. Thus, using methods such as that disclosed by Amsden microspheres having a very narrow diameter distribution are produced.

In an exemplary embodiment, the nucleic acid-containing subunit includes an acrylamide moiety. The nucleic acid-acrylamide conjugate is combined with bisacrylamide (3:1 w/w) in an aqueous buffer (e.g., sodium phosphate, pH 7.4). The solution is optionally deoxygenated by bubbling nitrogen gas through the solution. A catalyst, such as ammonium peroxodisulfate in water is added and the aqueous component is homogenized in an organic medium comprising a mixture of toluene and chloroform (4:1) containing a detergent, such as Pluroincs F-68. Polymerization of the resulting emulsion is initiated by the addition of N,N′,N″,N′˜5-tetramethylethylenediamine. The resulting suspension is stirred for approximately one-half hour and the phases are separated by centrifugation. The organic phase is removed and the microparticles located at the bottom of the aqueous phase are repeatedly washed with and aqueous buffer.

The resulting microparticles are characterized by methods well known in the art. Using standard techniques, the half-life of the particle in the blood can be determined and the distribution of the particles in the body is determined by, for example, injecting radioactively labeled microspheres and counting the individual organs after sacrifice of the experimental animal (Sj6holm et al., supra). The particle size is also determined by art recognized methods, such as scanning electron microscopy (Höglund et al., J. Gen. Virol. 21: 359 (1973)). Other methods of preparing and characterizing microparticles according to the present invention will be apparent to those of skill in the art.

The particles of the invention can be of substantially any useful size for a particular application. In a preferred embodiment, the particles range from about 0.001 μm to about 100 μm, more preferably from about 0.1 μm to about 10 μm in diameter.

(c). Tissue Targeted Agents and Promoters of Cellular Uptake

In the methods of the invention, the amount of transfection desired is that which will result in a therapeutic effect, i.e. prevention, palliation, and/or cure of an animal or human disease (“in vivo” gene therapy). Optionally, the carrier molecule and/or construct of the invention may provide for targeting and/or expression in a particular cell type or types.

The size, nature and specific sequence of the nucleic acid composition to be transferred to the target cell can be optimized for the particular application for which it is intended, and such optimization is well within the skill of the artisan in this field.

However, the nature of the target cells within the individual into which it is desired to transfer a nucleic acid composition, may have a significant bearing on the choice of the particular multifunctional molecular complex of the present invention. For example, where it is desired to transfer nucleic acid molecules to target cells by injecting them intramuscularly to evoke an immune response, it will be found that this transfer can be effected by use of a multifunctional molecular complex of the present invention, as defined above, comprising a cationic polyamine to which is attached, as the endosome membrane disruption promoting component, a lipophilic long chain alkyl group as defined above.

Another approach in the art to delivery of genetic material to target cells is one that takes advantage of natural receptor-mediated endocytosis pathways that exist in such cells. Several cellular receptors have been identified heretofore as desirable agents by means of which it is possible to achieve the specific targeting of drugs, and especially macromolecules and molecular conjugates serving as carriers of genetic material of the type with which the present invention is concerned. These cellular receptors allow for specific targeting by virtue of being localized to a particular tissue or by having an enhanced avidity for or activity in a particular tissue. See, e.g., J. L. Bodmer et al., Meth. Enzymol. 112: 298-306 (1985). This affords the advantages of lower doses or significantly fewer undesirable side effects.

One of the better known examples of a cell and tissue selective receptor is the asialoglycoprotein receptor present in hepatocytes. The asialoglycoprotein receptor is an extracellular receptor with a high affinity for galactose, especially tri-antennary oligosaccharides, i.e., those with three somewhat extended chains or spacer arms having terminal galactose residues; see, e.g., Lodish, TIBS 16: 374-77 (1991). This high affinity receptor is localized to hepatocytes and is not present in Kupffer cells; allowing for a high degree of selectivity in delivery to the liver.

It has also been proposed in the art of receptor-mediated gene transfer that in order for the process to be efficient in vivo, the assembly of the DNA complex should result in condensation of the DNA to a size suitable for uptake via an endocytic pathway. See, e.g., Perales et al., Proc. Nat. Acad. Sci. USA 91: 4086-4090 (1994).

An alternative method of providing cell-selective binding is to attach an entity with an ability to bind to the cell type of interest; commonly used in this respect are antibodies which can bind to specific proteins present in the cellular membranes or outer regions of the target cells. Alternative receptors have also been recognized as useful in facilitating the transport of macromolecules, such as biotin and folate receptors; see, Low et al., WO 90/12095, published 18 Oct. 1990; Low et al., WO 90/12096, published 18 Oct. 1990; Low et al., U.S. Pat. No. 5,108,921, Apr. 28, 1992; Leamon et al., Proc. Nat. Acad. Sci. USA 88: 5572-5576 (1991); transferrin receptors; insulin receptors; and mannose receptors (see further below). The enumerated receptors are merely representative, and other examples will readily come to the mind of the artisan.

The conjugation of different functionalities on the same molecule has also been utilized in the art. For example, in 1988, Wu et al., J. Biol. Chem. 263: 14621-14624 (1988) described a method for cellular receptor mediated delivery of DNA to hepatocytes. This method was further described in Wu et al., Bio Chem. 27: 887-892 (1988); Wu et al., U.S. Pat. No. 5,166,320, Nov. 24, 1992; and Wu et al., WO 92/06180, published 16 Apr. 1992. The method consists of attaching a glycoprotein, asialoorosomucoid, to poly-lysines to provide a hepatocyte selective DNA carrier. The function of the poly-lysine is to bind to the DNA through ionic interactions between the positively charged (cationic) epsilon amino groups of the lysines and the negatively charged (anionic) phosphate groups of the DNA. Orosomucoid is a glycoprotein, which is normally present in human serum. Removal of the terminal sialic acid (N-acetyl neuraminic acid) from the branched oligosaccharides exposes terminal galactose oligosaccharides, for which hepatocyte receptors have a high affinity, as already described.

After binding to the asialoglycoprotein receptor on hepatocytes, the protein is taken into the cell by endocytosis into a pre-lysosomal endosome. The DNA, ionically bound to the poly-lysine-asialoorosomucoid carrier, is also taken into the endosome.

Additional work using this delivery system, e.g., that done by Wilson et al., J. Biol. Chem. 267: 11483-11489 (1992), has found that partial hepatectomy improves the persistence of the expression of the DNA delivered into the hepatocytes. The transfer of the DNA into cells by this mechanism is also significantly enhanced by the addition of cationic lipids; see, e.g., Mack et al., Am. J. Med. Sei. 307: 138-143 (1994). The use of a specific asialoglycoprotein is not required to achieve binding to the asialoglycoprotein receptor; this binding can also be accomplished with high affinity by the use of small, synthetic molecules having a similar configuration. The carbohydrate portion can be removed from an appropriate glycoprotein and be conjugated to other macromolecules; see, e.g., Wood et al., Bioconj. Chem. 3: 391-396 (1992). By this procedure the cellular receptor-binding portion of the glycoprotein is removed, and the specific portion required for selective cellular binding can be transferred to another molecule.

There is an ample literature on the preparation of synthetic glycosides which can be attached to macromolecules and confer on them the ability to bind to the corresponding galactose specific receptor. The importance of branched glycosides was recognized early; see, Lee, Carb. Res. 67: 509-514 (1978). Further work delineated that sugar density (Kawaguchi et al., J. Biol. Chem. 256: 2230-2234 (1981)) and spacial relationships (Lee et al., J. Biol. Chem. 258: 199-202 (1983)) are important determinants of binding potency. Reductive amination of a peptide with a branched tri-lysine amino terminus gives a ligand ending with four galactosyl residues that can be readily coupled to poly-lysine or other macromolecules and has been used to prepare DNA constructs; see, Plank et al., Bioconj. Chem. 3: 533-539 (1992); Thiopropionate and thiohexanoate glycosidic derivatives of galactose have been prepared and linked to L-lysyl-L-lysine to form a synthetic tri-antennary galactose derivative. A bisacridine spermidine derivative containing this synthetic tri-antennary galactose has been used to target DNA to hepatocytes; see, Szoka et al., WO 93/19768, published 14 Oct. 1993; and Haensler et al., Bioconj. Chem. 4: 85-93 (1993).

Other means of providing cellular receptor based facilitation of gene transfer into cells using carriers have been described in the art. Antibodies specific for cell surface thrombomodulin have been used with poly-lysine as a delivery system for DNA in vitro and in vivo; see Trubetskoy et al., Bioconj. Chem. 3: 323-327 (1992). The transferrin receptor has also been used to target DNA to erythroblasts, K562 macrophages and ML-60 leukemic cells; see, Wagner et al., Proc. Nat. Acad. Sci. USA 87: 3410-3414 (1990); Zenke et al., Proc. Nat. Acad. Sci. USA 87: 3655-3659 (1990); and Citro et al., Proc. Nat. Acad. Sci. USA 89: 7031-7035 (1990). These studies used both small oliogodeoxynucleotides as well as large plasmids.

The ability of poly-lysine to facilitate DNA entry into cells is significantly enhanced if the poly-lysine is chemically modified with hydrophobic appendages; see Zhou et al., Biochim. Biophys. Acta 1189: 195-203 (1994); complexed with cationic lipids; see Mack et al., Am. J. Med. Sci. 307: 138-143 (1994) or associated with viruses. Many viruses infect specific cells by receptor-mediated binding and insertion of the viral DNA/RNA into the cell and thus this action of the virus is similar to the facilitated entry of DNA described above.

Replication-incompetent adenovirus has been used to enhance the entry of transferrin-poly-lysine complexed DNA into cells; see, Curiel et al., Proc. Nat. Acad. Sci. USA 88: 8850-8854 (1991); Wagner et al., Proc. Nat. Acad. Sci. USA 89: 6099-6103 (1992); Cotton et al., Proc. Nat. Acad. Sci. USA 89: 6094-6098 (1992); and Gao et al., Hum. Gene Ther. 4: 17-24 (1993). The adenovirus enhances the entry of the poly-lysine-transferrin-DNA complex when covalently attached to the poly-lysine and when attached through an antibody-binding site. There does not need to be a direct attachment of the adenovirus to the poly-lysine-transferrin-DNA complex, and it can facilitate the entry of the complex when present as a simple mixture. The poly-lysine transferrin-DNA complex provides receptor specific binding to the cells and is internalized into endosomes along with the DNA. Once inside the endosomes, the adenovirus facilitates entry of the DNA/transfenin-poly-lysine complex into the cell by disruption of the endosomal compartment with subsequent release of the DNA into the cytoplasm. Replication-incompetent adenovirus has also been used to enhance the entry of uncomplexed DNA plasmids into cells without the benefit of the cell receptor selectivity conferred by the poly-lysine-transferrin complex; see, Yoshimura et al., J. Biol. Chem. 268: 2300-2303 (1993).

Synthetic peptides such as the N-terminus region of the influenza hemagglutinin protein are known to destabilize membranes and are known as fusogenic peptides. Conjugates containing the influenza fusogenic peptide coupled to poly-lysine together with a peptide having a branched tri-lysine amino terminus ligand ending with four galactosyl residues have been prepared as facilitators of DNA entry into hepatocytes; see, Plank et al., Bioconj. Chem. 3: 533-539 (1992). These conjugates combine the asialoglycoprotein receptor mediated binding conferred by the tetra-galactose peptide, the endosomal disrupting abilities of the influenza fusogenic peptide, and the DNA binding of the poly-lysine. These conjugates deliver DNA into the cell by a combination of receptor mediated uptake and internalization into endosomes. This internalization is followed by disruption of the endosomes by the influenza fusogenic peptide to release the DNA into the cytoplasm. In a similar fashion, the influenza fusogenic peptide can be attached to poly-lysine and mixed with the transferrin-poly-lysine complex to provide a similar DNA carrier selective for cells carrying the transferrin receptor; see, Wagner et al., Proc. Nat. Acad. Sci. USA 89: 7934-7938 (1992). Synthetically designed peptides can also be used; for example the “GALA” peptides (Subbarao et al., J. Biol. Chem. 26: 2964-2972 (1987)) have been coupled to DNA carriers and an enhanced facilitated entry into cells was observed (Haensler et al., Bioconj. Chem. 4: 372-379 (1993)). The cationic amphipathic peptide gramicidin S can facilitate entry of DNA into cells (Legendre et al., Proc. Nat. Acad. Sci. USA 90: 893-897 (1993)), but also requires a phospholipid to achieve significant transfer of DNA.

Positively charged liposomes have also been widely used as carriers of DNA which facilitate entry into cells; see, e.g., Szoka et al., WO 93/19768, published 14 Oct. 1993; Debs et al., WO 93/24640, published 9 Dec. 1993; Felgner et al., WO 91/16024, published 31 Oct. 1991; Felgner et al., Nature 337: 387-388 (1989); Rose et al., BioTechniques 10: 520-525 (1991); Bennett et al., Mol. Pharm. 41: 1023-1033 (1992); Felgner et al., J. Biol. Chem. 269: 2550-2561 (1994); Smith et al., Biochim. Biophys. Acta 1154: 327-340 (1993). These carrier compositions have also included pH sensitive liposomes; see, Chu et al., Pharm. Res. 7: 824-854 (1990); Legendre et al., Pharm. Res. 9: 1253-1242 (1992).

A poly-cationic lipid has been prepared by coupling dioctadecylamidoglycine and dipalmitoyl phosphatidylethanolamine to a 5-carboxyspermine; see, Behr et al., Proc. Nat. Acad. Sci. USA 86: 6982-6986 (1989); Barthel et al., DNA and Cell Biol. 12: 553-560 (1993); Loeffler et al., Meth. Enzymol. 217: 599-618 (1993); Behr et al., U.S. Pat. No. 5,171,678, Dec. 15, 1992. These lipophilic-spermines are very active in transferring DNA through cellular membranes.

Combinations of lipids have been used to facilitate the transfer of nucleic acids into cells. For example, in U.S. Pat. No. 5,283,185 there is disclosed such a method, which utilizes a mixed lipid dispersion of a cationic lipid with a co-lipid in a suitable solvent. The lipid has a structure, which includes a lipophilic group derived from cholesterol, a linker bond, a linear alkyl spacer arm, and a cationic amino group; and the co-lipid is phosphatidylcholine or phosphatidylethanolamine.

Macrophages have receptors for both mannose and mannose-6-phosphate, which can bind to and internalize molecules displaying these sugars. The molecules are internalized by endocytosis into a pre-lysosomal endosome. This internalization has been used to enhance entry of oligonucleotides into macrophages using bovine serum albumin modified with mannose-6-phosphate and linked to an oligodeoxynucleotide by a disulfide bridge to a modified 3′ end; see, Bonfils et al., Nucl. Acids Res. 20: 4621-4629 (1992). Similarly, oligodeoxynucleotides modified at the 3′ end with biotin were combined with mannose-modified streptavidin, and were also found to have facilitated entry into macrophages; see, Bonfils et al., Bioconj. Chem. 3: 277-284 (1992).

Various peptides and proteins, many of which are naturally occurring, have been shown to have receptors on cell surfaces that once they are attached thereto, allow them to become internalized by endocytosis. Materials bound to these receptors are delivered to endosomal compartments inside the cell. Examples include insulin, vasopressin, low-density lipoprotein, epidermal growth factor and others. This internalization has also been used to facilitate entry of DNA into cells; e.g., insulin has been conjugated to polylysine to provide facilitated DNA entry into cells possessing an insulin receptor; see, Huckett et al., Biochem. Pharmacol. 40: 253-263 (1990).

In another preferred embodiment, the present invention provides compositions that include a moiety that enhances cellular uptake. Many of the moieties discussed above not only aid in targeting the micleic acids to a particular cell, but also enhance the uptake of the constructs by the cell. Moreover, delivery vehicles in which the composition of the invention is dissolved and/or suspended are know that enhance cellular uptake. Examples of such a delivery vehicles are provided in Edwards et al., U.S. Pat. No. 5,985,320, Nov. 16, 1999.

2. Pharmaceutical Formulations

The invention also provides pharmaceutical compositions comprising one or more compounds of this invention in association with a pharmaceutically acceptable carrier. Preferably these compositions are in unit dosage forms such as tablets, pills, capsules, powders, granules, sterile parenteral. solutions or suspensions, metered aerosol or liquid sprays, drops, ampoules, auto-injector devices or suppositories; for oral, parenteral, intranasal, sublingual. or rectal administration, or for administration by inhalation or insufflation. Alternatively, the compositions may be presented in a form suitable for once-weekly or once-monthly administration; for example, an insoluble salt of the active compound, may be adapted to provide a depot preparation for intramuscular injection. An erodible polymer containing the active ingredient may be envisaged. For preparing solid compositions such as tablets, the principal active ingredient is mixed with a pharmaceutical carrier, e.g. conventional tableting ingredients such as corn starch, lactose, sucrose, sorbitol, talc, stearic add, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof.

When referring to these preformulation compositions as homogeneous, it is meant that the compound of the invention is dispersed substantially evenly throughout the composition so that the composition may be readily subdivided into equally effective unit dosage forms such as tablets, pills and capsules.

The liquid forms in which the novel compositions of the present invention may be incorporated for administration orally or by injection include aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium carboxymethyl cellulose, methylcellulose, polyvinyl-pyrrolidone or gelatin.

Any physiologically acceptable medium may be employed for administering the compositions of the invention, such as deionized water, saline, phosphate-buffered saline, 5% dextrose in water, and the like, depending upon the route of administration. Other components may be included in the formulation such as buffers, stabilizers, biocides, etc. These components have found extensive exemplification in the literature and need not be described in particular here.

3. Methods

The present invention further provides methods for delivering a nucleic acid composition into a cell. The method comprises the step of contacting cells of said individual with a multifunctional molecular complex of the present invention, which includes said nucleic acid composition. Here again, the nucleic acid molecule comprises a nucleotide sequence that either encodes a desired peptide or protein, or serves as a template for functional nucleic acid molecules. The micleic acid molecule is administered free from retroviral particles. The desired protein may either be a protein which functions within the individual or serves to initiate an immune response.

The nucleic acid molecule may be administered to the cells of said individual on either an in vivo or ex vivo basis, ie., the contact with the cells of the individual may take place within the body of the individual in accordance with the procedures which are most typically employed, or the contact with the cells of the individual may take place outside the body of the individual by withdrawing cells which it is desired to treat from the body of the individual by various suitable means, followed by contacting of said cells with said nucleic acid molecule, followed in turn by return of said cells to the body of said individual.

The method of transferring a nucleic acid composition to the cells of an individual provided by the present is invention, includes particularly a method of immunizing an individual against a pathogen. In this method, the nucleic acid composition administered to said cells, comprises a nucleotide sequence that encodes a peptide which comprises at least an epitope identical to, or substantially similar to an epitope displayed on said pathogen as antigen, and said nucleotide sequence is operatively linked to regulatory sequences. The nucleic acid molecule must, of course, be capable of being expressed in the cells of the individual.

The method of transferring a nucleic acid composition to the cells of an individual provided by the present invention, further includes methods of immunizing an individual against a hyperproliferative disease or an autoimmune disease. In such methods, the nucleic acid composition administered to the cells of the individual includes a nucleotide sequence that encodes a peptide that comprises at least an epitope identical to or substantially similar to an epitope displayed on a hyperproliferative disease-associated protein or an autoimmune disease-associated protein, respectively, and is operatively linked to regulatory sequences. Here again, the nucleic acid molecule must be capable of being expressed in the cells of the individual.

The subject may be any mammal, particularly a mammal having symptoms of a genetically-based disorder. Thus, the subject application finds use in domestic animals, feed stock, such as bovine, ovine, and porcine, as well as primates, particularly humans. The mammalian host may be pregnant, and the intended recipient of the gene-based therapy may be either the gravid female or the fetus or both. In the method of the invention, transfection in vivo is obtained by introducing a therapeutic transcription or expression vector into the mammalian host, either as naked DNA or complexed to lipid carriers, particularly cationic lipid carriers. The constructs may provide for integration into the host cell genome for stable maintenance of the transgene or for episomal expression of the transgene. The introduction into the mammalian host may be by any of several routes, including intravenous or intraperitoneal injection, intratracheally, intrathecally, parenterally, intraarticularly, intramuscularly, etc. Of particular interest is the introduction of a therapeutic expression vector into a circulating bodily fluid. Thus, iv administration and intrathecal administration are of particular interest since the vector may be widely disseminated following such a route of administration.

The amount of the compositions of the invention used will be sufficient to provide for adequate dissemination to a variety of tissues after entry of the DNA or complexes into the bloodstream and to provide for a therapeutic level of expression in transfected tissues. A therapeutic level of expression is a sufficient amount of expression to, prevent, treat or palliate a disease of the host mammal. In addition, the dose of the plasmid DNA expression vector used must be sufficient to produce significant levels of transgene expression in multiple tissues in vivo for example, I mg of an expression plasmid alone is injected into a mouse to achieve high level expression of the CAT gene in multiple tissues. Other DNA. sequences, such as adenovirus VA genes can be included in the administration medium and. be co-transfected with the gene of interest. The presence of genes coding for the adenovirus VA gene product may significantly enhance the translation of mRNA transcribed from the plasmid.

The level and tissues of expression of the recombinant gene may be determined at the mRNA level and/or at the level of polypeptide or protein. Gene product may be quantitated by measuring its biological activity in tissues. For example, enzymatic activity can be measured by biological assay or by identifying the gene product in transfected cells by immunostaining techniques such as probing with an antibody which specifically recognizes the gene product or a reporter gene product present in the expression cassette. Alternatively, potential therapeutic effects of the gene product can be measured, for example where the DNA sequence of interest encodes GM-CSF, by determining the effects of gene expression on survival of lethally irradiated animals in which the GM-CSF transgene is expressed. Production of significant amounts of a transgene product will substantially prolong the survival of these mice.

Where expression of the polypeptide/protein or even the mRNA itself confers a changed biochemical phenotype upon the host, the presence of a new phenotype or absence of an old phenotype may be evaluated; for example, as a result of transfection of the host cells, there may be enhanced production of pre-existing desirable products formerly produced in insufficient quantities or there may be reduction or even suppression of an undesirable gene product using antisense, ribozyme or co-suppression technologies; in the case of suppression, a reduction of the gene product may be determined. Typically, the therapeutic cassette is not integrated into the host cell genome. If necessary, the treatment can be repeated on an ad hoc basis depending upon the results achieved. If the treatment is repeated, the mammalian host can be monitored to ensure that there is no adverse immune response to the treatment.

The subject compositions can be provided for use in one or more procedures. Kits will usually include the DNA either as naked DNA bearing the reactive linker group or already tethered to carriers. Additionally, lipid carriers may be provided in a separate container for complexing with the provided DNA. The DNA either for direct injection or for complexing with lipid carriers, or the lipid carrier/DNA complexes may be present as concentrates which may be further diluted prior to use or they may be provided at the concentration of use, where the vials may include one or more dosages. Conveniently, single dosages may be provided in syringes, contained in sterilized containers, so that the physicians or veterinarian may employ the syringes directly, where the syringes will have the desired amount and concentration of agents. Thus, the kit may have a plurality of syringes containing the DNA or the DNA/lipid carrier complexes in appropriate proportional amounts. When the syringes contain the formulation for direct use, usually there will be no need for other reagents for use with the method.

The invention finds use in in vivo prevention, treatment and/or palliation of a number of diseases. In vivo replacement of a gene can be accomplished by techniques such as homologous recombination or initial knockout of the aberrant gene and subsequent replacement with the desired transgene.

Thus, in accordance with the present invention there is provided a method for the transfer of a nucleic acid composition to target cells on an in vitro basis. In this method target cells are contacted with a multifunctional molecular complex which includes said nucleic acid composition. In one embodiment, the target cells have been isolated from an individual, and all of the cells are thus of the same type, and it is not necessary, therefore, for the complex to include a receptor specific binding component. An especially preferred embodiment is one in which a microorganism culture is maintained under fermentation conditions, and a protein product is expressed by the microorganism as a result of the transfer thereto of nucleic acid compositions using the multifunctional molecular complex of the present invention. The protein product is isolated and purified. Here again, a single type of target cell is involved, so that it is not necessary that a receptor specific binding component be present.

This method provides for transfer to target cells of a nucleic acid molecule that comprises a micleotide sequence that either encodes a desired peptide or protein, or serves as a template for functional micleic acid molecules. The desired protein or functional nucleic acid molecule may be any product of industrial, commercial or scientific interest, e.g., therapeutic agents including vaccines; foodstuffs and nutritional supplements; compounds of agricultural significance such as herbicides and plant growth regulants, insecticides, miticides, rodenticides, and fungicides; compounds useful in animal health such as parasiticides including nematocides; and so forth. The target cells are typically cultures of host cells comprising microoganism cells such as bacteria and yeast, but may also include plant and mammalian cells. The cell cultures are maintained in accordance with fermentation techniques well known in the art, which maximize production of the desired protein or functional nucleic acid molecule, and the fermentation products are harvested and purified by known methods.

It is understood that the examples and embodiments described herein are for illustrative purposes only and that various modifications or changes in light thereof will be suggested to persons skilled in the art and are to included within the spirit and purview of this application and are considered within the scope of the appended claims.

All publications, patents, and patent applications cited herein are hereby incorporated by reference in their entirety for all purposes.

1. A substantially water-soluble polymer comprising a first subunit comprising a first nucleic acid and an ethylene-containing moiety, the first subunit joined to a framework selected from liposomes, micelles, colloids, proteins, lipids dendrimers, protein aggregates, modified cells, modified viral particles and silica beads.

2. The polymer according to claim 1, wherein said first subunit is non-covalently attached to the framework.

3. The polymer according to claim 1, further comprising a cleavable moiety.

4. The polymer according to claim 3, wherein said cleavable moiety is located between said first subunit and said framework,

5. The polymer according to claim 3, wherein said cleavable moiety is a member selected from groups cleaved by change in pH, enzymatic action, reduction, oxidation, light, heat and combinations thereof

6. The polymer according to claim 5, wherein said cleavable moiety is cleaved by a process occurring in a biological system.

7. The polymer according to claim 6, wherein said cleavable moiety is a member selected from disulfides, esters, phosphodiesters and combinations thereof.

8. The polymer according to claim 1, wherein said first subunit further comprises a linker group adjoining said first nucleic acid and said ethylene-containing moiety.

9. (canceled)

10. (canceled)

11. (canceled)

12. (canceled)

13. The polymer according to claim 1, wherein said ethylene-containing moiety comprises a member selected from —CH₂═CHX¹, —CH₂═CX²Y¹ and combinations thereof, wherein
X¹, X² and Y¹ are members independently selected from H, (═O), —NR¹R², —OH, and —OR³, wherein
R¹, R² and R³ are members independently selected from H, alkyl, substituted alkyl, aryl and substituted aryl.

14. (canceled)

15. (canceled)

16. The polymer according to claim 13, wherein at least one of R¹, R² and R³ comprises a moiety selected from poly(ethyleneglycol), poly(propyleneglycol) and combinations thereof.

17. A polymer comprising a first subunit comprising a first nucleic and an ethylene-containing moiety, the first subunit joined to a framework, the framework comprising either (a) a homopolymer or copolymer of a monomer selected from acrylate, C₁-C₆ alkylacrylate, methylmethacrylate, triethyleneglycolmethacrylate, poly(ethyleneglycol)methacrylate, hydroxyethylmethacrylate, glycerylmethacrylate, vinyl alcohol, ethylcyanoacrylate and combinations thereof or (b) a homopolymer or copolymer selected from poly(ethylene glycol), polyethylene oxide, poly(aminoacid), poly(glutamic acid), poly(aspartic acid), poly(lactic acid), poly(glycolic acid), poly(succinimide), poly(esters), poly(carbohydrates), polyols, poly(ethers), polyamines, chondroitin sulfate, crosslinked liposomes, poly (N-vinylpyrrolidone), poly(ethylene-vinyl acetate), poly(urethanes), poly(maleic acid homo- or co-polymer), hyalouronic acid, poly(anhydrides) and poly(vinyl alcohols).

18. The polymer according to claim 1, further comprising a tissue-specific targeting moiety or a moiety that enhances cellular uptake.

19. (canceled)

20. (canceled)

21. (canceled)

22. (canceled)

23. (canceled)

24. (canceled)

25. (canceled)

26. (canceled)

27. The polymer according to claim 1, wherein said polymer is a copolymer of said first subunit and a second subunit, the second subunit having a sequence different from the of said first subunit, and further wherein the second subunit may comprise a third nucleic acid, the nucleic acid optimally having a sequence different from that of said first nucleic acid.

28. (canceled)

29. (canceled)

30. A polymer comprising a first subunit comprising a first nucleic acid and an ethylene-containing moiety, the first subunit covalently or non-covalently joined to a polymeric framework and further wherein the polymeric particle further comprises a tissue-specific targeting moiety.

31. (canceled)

32. (canceled)

33. (canceled)

34. (canceled)

35. (canceled)

36. (canceled)

37. (canceled)

38. (canceled)

39. (canceled)

40. The polymer according to claim 30, wherein said polymeric framework comprises either (a) a polymer derived from a member selected from acrylate, acrylamide, C₁-C₆ alkylacrylate, (alkyl)acrylamide, methylmethacrylate, triethyleneglycolmethacrylate, poly(ethyleneglycol)methacrylate, hydroxyethylmethacrylate, glycerylmethaerylate, vinyl alcohol, ethylcyanoacrylate and combinations thereof; or (b) a member selected from liposomes, micelles, colloids, sugars, proteins, lipids, nucleic acids dendrimers, protein aggregates, modified cells, modified viral particles, peptides, polysaccharides and silica beads; or (c) a homopolymer or copolymer selected from poly(ethylene glycol), polyethylene oxide, poly(aminoacid), poly(glutamic acid), poly(aspartic acid), poly(lactic acid), poly(glycolic acid), poly(succinimide), poly(esters), polysaccharides, poly(carbohydrates), polyols, poly(ethers), polyamines, chondroitin sulfate, crosslinked liposomes, peptides, dextran derivatives, poly (N-vinylpyrrolidone), poly(ethylene-vinyl acetate), poly(urethanes), poly(maleic acid homo- or co-polymer), hyalouronic acid, poly(glycerol), starch, poly(anhydrides), poly(vinyl alcohols), and poly(orthoesters).

41. (canceled)

42. The polymer according to claim 30, further comprising a moiety that enhances cellular uptake.

43. (canceled)

44. The polymer according to claim 30, wherein said first nucleic acid is hybridized to a second nucleic acid.

45. The polymer according to claim 44, wherein said first and second nucleic acids are independently selected from single-stranded or double stranded nucleic acids.

46. (canceled)

47. (canceled)

48. (canceled)

49. (canceled)

50. (canceled)

51. (canceled)

52. (canceled)

53. (canceled)

54. (canceled)

55. The polymer according to claim 30, further comprising a bioactive compound encapsulated by said polymer.

56. A pharmaceutical formulation comprising a pharmaceutically acceptable carrier and the substantially water-soluble polymer of claim 1.

57. (canceled)

58. (canceled)

59. (canceled)

60. (canceled)

61. (canceled)

62. (canceled)

63. (canceled)

64. (canceled)

65. (canceled)

66. The polymer according to claim 30, wherein the tissue-specific targeting moiety is a cell and tissue selective receptor.

67. The polymer according to claim 66, wherein the cell and tissue selective receptor is an asialoglycoprotein receptor and the tissue-specific targeting moiety is:
(i.) capable of selectively binding to a targeted tissue;
(ii.) an antibody;
(iii.) a transport macromolecule facilitator;
(iv.) a biotin or folate receptor, transferring receptor, insulin receptor, or a mannonse receptor; or
(v.) a hepatocyte selective DNA carrier.

68. A polymeric material comprising:
(i.) a homopolymer or a copolymer;
(ii.) a first subunit precursor comprising a first nucleic acid and an ethylene-containing moiety, the first subunit precursor being covalently or non-covalently linked to the homopolymer or copolymer; and
(iii.) a bioactive compound encapsulated by the homopolymer or copolymer

69. A method for introducing a drug into a mammalian host, which comprises introducing into a circulating body fluid, organ, cells or body cavity of the host a construct of a first subunit of an oligonucleotide modified with an ethylene-containing moiety, the first subunit being incorporated into or joined to a framework.

70. A method for changing in vivo a genotype and/or modulating the phenotype of cells in tissues of a mammalian host, the method comprising introducing into a circulating body fluid, organ, cells or body cavity of the host a construct of a first subunit comprising a first nucleic acid and an ethylene-containing moiety, the first subunit joined to or incorporated into a framework.

71. A method for introducing a modified polynucleotide into an eukaryotic-cell in a living animal, the method comprising contacting the cell with a construct of an oligonucleotide modified with an ethylene-containing moiety, the modified oligonucleotide being incorporated into or joined to a framework.
